(19) United States
(12) Patent Application Publication
(10) Pub. No.: US 2006/0100224 A1
(43) Pub. Date: May 11, 2006

(54) Piperidine ouracil used as a medicament for treating bacterial infections

(76) Inventors: Niels Svenstrup, Wuppertal (DE); Alexander Kuhl, Hagen (DE); Dietmar Flubacher, Freiburg (DE); Michael Brands, Hamden, CT (US); Kerstin Ehlert, Velbert (DE); Christoph Ladel, Ivrea (IT); Michael Otteneder, Arlesheim (CH); Jorg Keldenich, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/505,983

(22) PCT Filed: Feb. 13, 2003
(86) PCT No.: PCT/EP03/01405

§ 371 Date: Dec. 23, 2005

(30) Foreign Application Priority Data
Feb. 26, 2002 (DE) 102 08 256.1

(51) Int. Cl. A61K 31/513; C07D 403/02

(52) U.S. Cl. 514/269; 544/310

(57) ABSTRACT

The invention relates to piperidine ouracil and a method for the production thereof in addition to the use thereof in the production of medicaments for treating and/or in the prophylaxis of diseases, especially bacterial diseases.

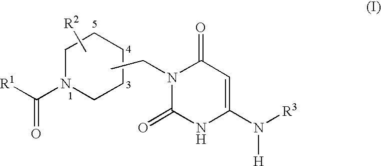

The invention relates to piperidinouracils and process for their preparation, and to their use for producing medicaments for the treatment and/or prophylaxis of diseases, especially of bacterial diseases.

Gram-positive eubacteria contain three different DNA polymerase exonucleases which are referred to as Pol 1, Pol 2 and Pol 3. Pol 3 is an enzyme which is necessary for the replicative synthesis of DNA.

The suitability of uracil derivatives for the treatment of bacterial infections has already been known for some time. Thus, WO 01/29010 describes 3-aminocarbonyl-substituted phenylaminouracils, WO 96/06614 describes 3-alkylidene-substituted uracils, WO 00/71523 describes 3-alkanoyloxyalkyluracils and WO 00/20556 describes uracils with zinc finger-active unit as antibacterial compounds.

J. Med. Chem., 1999, 42, 2035, Antimicro. Agents and Chemotherapy, 1999, 43, 1982 and Antimicro. Agents and Chemotherapy, 2000, 44, 2217 describe phenylaminouracils as antibacterial compounds.

One object of the present invention is to provide novel, alternative soluble compounds with antibacterial effect for the treatment of bacterial diseases in humans and animals.

The present invention relates to compounds of the formula (I), 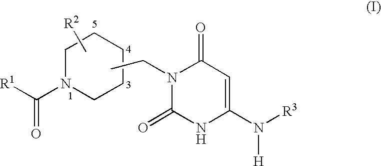
in which

R¹ is heteroaryl,
where heteroaryl may be substituted by 0, 1, 2 or 3 substituents selected independently of one another from the group consisting of alkyl, alkoxy, alkylthio, halogen, alkanoyl, hydroxy, trifluoromethyl, trifluoromethoxy, nitro, amino, alkylamino, alkanoylamino, cyano, carboxy, cycloalkyl, heterocyclyl, aryl and optionally methyl-substituted heteroaryl,

R² is hydrogen or alkyl,

R³ is a substituent of the following formula 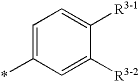
in which
   R³⁻¹ and R³⁻² are selected independently of one another from the group consisting of alkyl, alkenyl, alkynyl, alkylthio, cycloalkyl and halogen, or
   R³⁻¹ and R³⁻² form together with the carbon atoms to which they are bonded a cycloalkyl or heterocyclyl ring which is optionally substituted by up to 3 halogen.

The compounds of the invention may also be in the form of their salts, solvates or solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Physiologically acceptable salts of the compounds (I) also include salts of conventional bases, such as by way of example and preferably alkali metal salts (e.g. sodium and potassium salts), alkaline earth metal salts (e.g. calcium and magnesium salts) and ammonium salts derived from ammonia or organic amines having 1 to 16 C atoms, such as by way of example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabiethylamine, arginine, lysine, ethylenediamine and methylpiperidine.

Solvates refers for the purposes of the invention to those forms of the compounds which form a complex in the solid or liquid state through coordination with solvent molecules. Hydrates are a specific form of solvates in which the coordination takes place with water.

For the purposes of the present invention, the substituents have the following meaning unless specified otherwise:

Alkyl per se and “alk” and “alkyl” in alkoxy, alkylamino alkylthio, alkanoyl and alkanoylamino stand for a linear or branched alkyl radical having ordinarily from 1 to 6, preferably 1 to 4, particularly preferably 1 to 3, carbon atoms, by way of example and preferably methyl, ethyl, n-propyl, isopropyl, tert-butyl, n-pentyl and n-hexyl.

Alkenyl stands for a straight-chain or branched alkenyl radical having 2 to 6 carbon atoms. Preference is given to a straight-chain or branched alkenyl radical having 2 to 4, particularly preferably having 2 to 3 carbon atoms. Preferred examples which may be mentioned are: vinyl, allyl, n-prop-1-en-1-yl and n-but-2-en-1-yl.

Alkynyl stands for a straight-chain or branched alkenyl radical having 2 to 6 carbon atoms. Preference is given to a straight-chain or branched alkenyl radical having 2 to 4, particularly preferably having 2 to 3 carbon atoms. Preferred examples which may be mentioned are: n-prop-1-yn-1-yl and n-but-2-yn-1-yl.

Alkoxy stands by way of example and preferably for methoxy, ethoxy, n-propoxy, isopropoxy, tert-butoxy, n-pentoxy and n-hexoxy.

Alkylamino stands for an alkylamino radical having one or two alkyl substituents (chosen independently of one another),, by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkylthio stands by way of example and preferably for methylthio, ethylthio, n-propylthio, isopropylthio, tert-butylthio, n-pentylthio and n-hexylthio.

Alkanoyl stands by way of example and preferably for acetyl and ethylcarbonoyl.

Alkanoylamino stands by way of example and preferably for acetylamino and ethylcarbonylamino.

Cycloalkyl stands for a cycloalkyl group having ordinarily 3 to 8, preferably 5 to 7, carbon atoms, by way of example and preferably cyclopropyl, cyclobutyl, cyclopentyl, cyclohexyl and cycloheptyl.

Aryl stands for a mono- to tricyclic aromatic, carbocyclic radical ordinarily having from 6 to 14 carbon atoms; by way of example and preferably phenyl, naphthyl and phenanthrenyl.

Heteroaryl stands for an aromatic, mono- or bicyclic radical ordinarily having from 5 to 10, preferably 5 to 6 ring atoms and up to 5, preferably up to 4, in particular up to 3, heteroatoms from the series S, O and N, by way of example and preferably thienyl, furyl, pyrrolyl, thiazolyl, oxazolyl, isoxazolyl, imidazolyl, pyridyl, pyrimidyl, pyridazinyl, indolyl, indazolyl, benzofuranyl, benzothiophenyl, quinolinyl, isoquinolinyl.

Heterocyclyl stands for a mono- or polycyclic, preferably mono- or bicyclic, nonaromatic heterocyclic radical ordinarily having from 4 to 10, preferably 5 to 8, in particular 5 or 6, ring atoms and up to 3, preferably up to 2, heteroatoms and/or hetero groups from the series N, O, S, SO, SO₂. The heterocyclyl radicals may be saturated or partially unsaturated. Preference is given to 5- to 6-membered, monocyclic saturated heterocyclyl radicals having up to two heteroatoms from the series O, N and S, such as by way of example and preferably tetrahydrofuran-2-yl, pyrrolidin-2-yl, pyrrolidin-3-yl, pyrrolinyl, piperidinyl, morpholinyl, perhydroazepinyl.

Halogen stands for fluorine, chlorine, bromine and iodine.

A symbol * on a bond denotes the point of linkage in the molecule.

If radicals in the compounds of the invention are substituted, the radicals may, unless otherwise specified, have one or more identical or different substituents. Substitution by up to three identical or different substituents is preferred. Substitution by one substituent is very particularly preferred.

Preference is given for the purposes of the present invention to compounds of the general formula (I)

in which

the uracil ring is linked via positions 3, 4 or 5 to the piperidine ring,

R¹ is heteroaryl,
where heteroaryl may be substituted by 0, 1, 2 or 3 substituents selected independently of one another from the group consisting of alkyl, alkoxy, alkylthio, halogen, hydroxy, carboxy, heterocyclyl and aryl,

R² is hydrogen or alkyl,

R³ is a substituent of the following formula 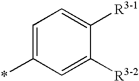
in which
   R³⁻¹ and R³⁻² are selected independently of one another from the group consisting of C₁-C₆-alkyl, C₂-C₆-alkenyl, C₂-C₆-alkynyl, C₁-C₆-alkylthio, C₃-C₆-cycloalkyl and halogen, or
   R³⁻¹ and R³⁻² form together with the carbon atoms to which they are bonded a C₃-C₆-cycloalkyl or heterocyclyl ring which is optionally substituted by up to 3 halogen.

Preference is given for the purposes of the present invention to compounds of the general formula (I),

in which

the uracil ring is linked via positions 3, 4 or 5 to the piperidine ring,

R¹ is heteroaryl,
where heteroaryl may be substituted by 0, 1, or 2 substituents selected independently of one another from the group consisting of alkyl, halogen and carboxy,

R² is hydrogen, and

R³ is a substituent of the following formula 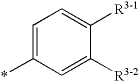
in which
   R³⁻¹ and R³⁻² are selected independently of one another from the group consisting of methyl, ethyl, fluorine and chlorine, or
   R³⁻¹ and R³⁻² form together with the carbon atoms to which they are bonded a C₅-cycloalkyl ring which is optionally substituted by up to 2 substituents selected independently of one another from the group consisting of chlorine or fluorine.

Preference is also given for the purposes of the present invention to compounds of the general formula (I) in which the uracil ring is linked via positions 3, 4 or 5 to the piperidine ring.

Preference is also given for the purposes of the present invention to compounds of the general formula (I) in which R¹ is a 5- to 6-membered heteroaryl radical, in particular a 5-membered heteroaryl radical.

Preference is also given for the purposes of the present invention to compounds of the general formula (I) in which R¹ is isoxazolyl or furyl.

Preference is also given for the purposes of the present invention to compounds of the general formula (I) in which R² is hydrogen.

Preference is also given for the purposes of the present invention to compounds of the general formula (I) in which

R³ is selected from the group of

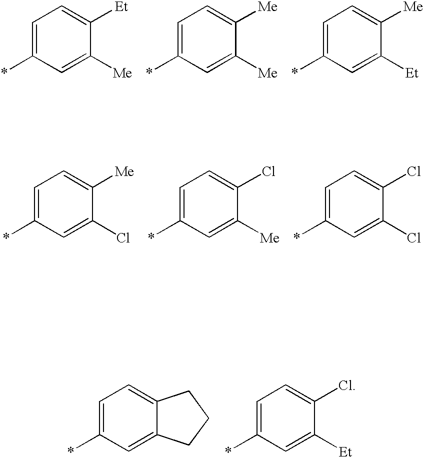

Preference is given for the purposes of the present invention among these in particular to compounds of the general formula (I) in which

R³ is selected from the group of

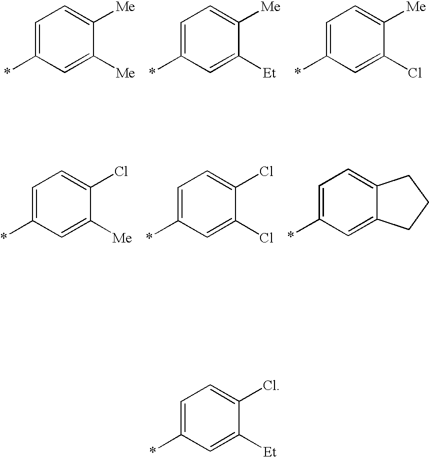

Preference is also given for the purposes of the present invention to compounds of the general formula (I) in which R³ is selected from the group of

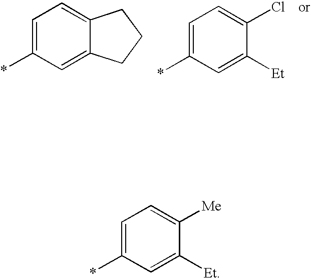

The present invention further relates to a process for preparing the compounds of the general formula (I), where compounds of the general formula 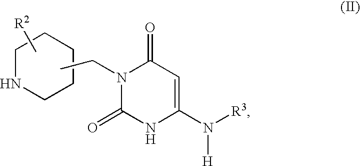
in which

R² and R³ have the meaning indicated above,
are reacted with compounds of the general formula 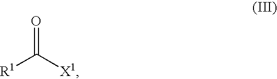
in which

R¹ has the meaning indicated above, and

X¹ is halogen, preferably chlorine or bromine, or hydroxy.

In the case where X¹ is halogen, the reaction takes place in inert solvents, if appropriate in the presence of a base, preferably in a temperature range from 0° C. to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, 2-butanone, dimethyl sulfoxide, acetonitrile or pyridine, with preference for dioxane or methylene chloride.

Examples of bases are alkali metal hydroxides such as sodium or potassium hydroxide, or alkali metal carbonates such as cesium carbonate, sodium or potassium carbonate, or amides such as lithium diisopropylamide, or other bases such as DBU, triethylamine or diisopropylethylamine, preferably diisopropylethylamine or triethylamine.

In the case where X¹ is hydroxy, the reaction takes place in inert solvents in the presence of conventional condensing agents, where appropriate in the presence of a base, preferably in a temperature range of from room temperature to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, dimethyl sulfoxide, acetonitrile or pyridine, with preference for tetrahydrofuran, dimethylformamide, 1,2-dichloroethane or methylene chloride.

Examples of conventional condensing agents are carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium-3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxaolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or mixtures thereof.

Examples of bases are alkali metal carbonates such as, for example, sodium or potassium carbonate, or sodium or potassium bicarbonate, or organic bases such as trialkylamines, e.g. triethylamine, N-methylmorpholine, N-methylpiperidine, 4-dimethylaminopyridine or diisopropylethylamine.

The combination of N-(3-dimethylaminoisopropyl)-N′-ethylcarbodiimide hydrochloride (EDC), 1-hydroxybenzotriazole (HOBt) and triethylamine in dimethylformamide, or benzotriazol-1-yloxytris(dimethylamino)phosphonium hexafluorophosphate (BOP) and diisopropylethylamine in tetrahydrofuran, is particularly preferred.

The compounds of the formula (III) are known or can be prepared in analogy to known processes.

One process for preparing compounds of the general formula (II) is characterized in that compounds of the formula 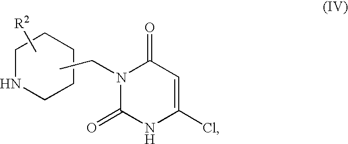
in which

R² has the meaning indicated above,
are reacted with compounds of the general formula
   H₂N—R³ (V)
   in which

R³ has the meaning indicated above,
where appropriate in the presence of a base.

The reaction takes place where appropriate in inert solvents, preferably in a temperature range from 100° C. to 150° C. under atmospheric pressure.

Examples of inert solvents are halohydrocarbons such as ethers such as dioxane, glycol dimethyl ether or diethylene glycol dimethyl ether, hydrocarbons such as benzene, xylene or toluene, or other solvents dimethylformamide, dimethylacetamide, dimethyl sulfoxide, pyridine or 1-methylpyrrolidinone, with preference for dioxane or 1-methylpyrrolidinone.

Examples of bases are alkali metal hydroxides such as sodium or potassium hydroxide, or alkali metal carbonates such as cesium carbonate, sodium or potassium carbonate, or amides such as lithium diisopropylamide, or other bases such as DBU, triethylamine or diisopropylethylamine, preferably diisopropylethylamine or triethylamine.

The compounds of the formula (V) are known or can be prepared in analogy to known processes.

One process for preparing the compounds of the general formula (IV) is characterized in that compounds of the formula 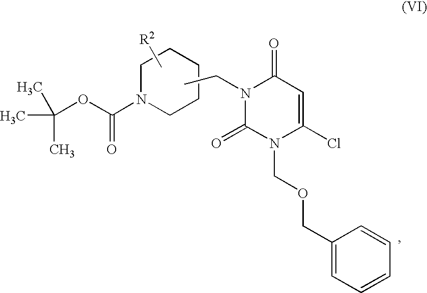
in which

R² has the meaning indicated above,

are reacted with trifluoroacetic acid, where appropriate in the presence of methylene chloride, preferably in a temperature range from 60° C. to reflux of the solvent under atmospheric pressure.

One process for preparing the compounds of the general formula (VI) is characterized in that the compound of the formula 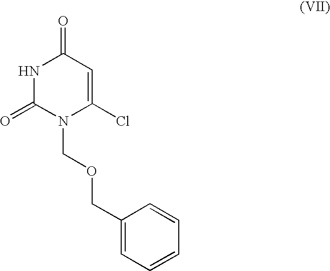
with compounds of the general formula 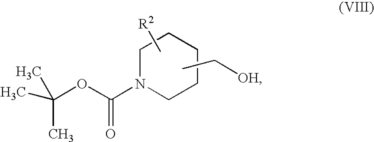
in which

R² has the meaning indicated above,

under Mitsunobu conditions in the presence of a phosphine and of a dialkyl azodicarboxylate, preferably in the presence of triphenylphosphine and diethyl azodicarboxylate, in tetrahydrofuran at 0° C. to room temperature under atmospheric pressure.

The compounds of the formula (VII) and (VIII) are known or can be prepared in analogy to known processes.

The preparation routes are to be illustrated by way of example by the following diagrams:

### Preparation of alkylpiperidinyl-6-chlorouracils

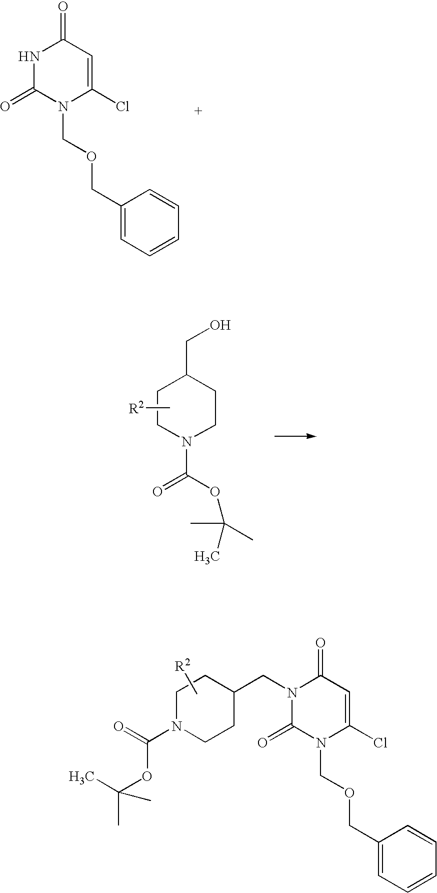

### Preparation of deprotected alkylpiperidinyl-6-chlorouracils

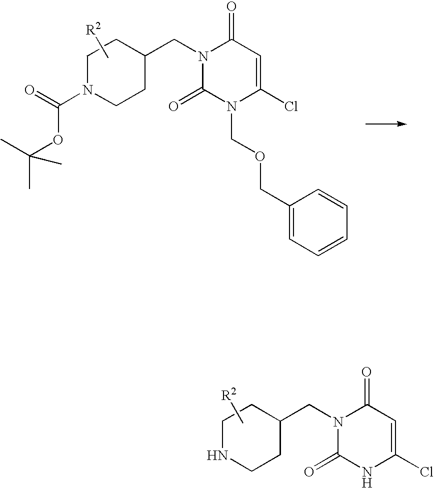

### Preparation of 6-aminoaryl-alkylpiperidinyluracils

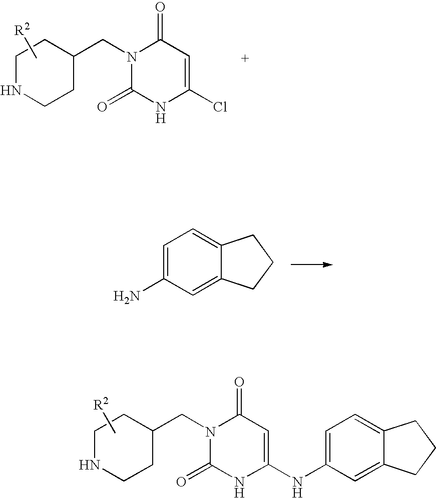

### Preparation of 6-aminoaryl-alkyl-N′-acylpiperidinyluracils

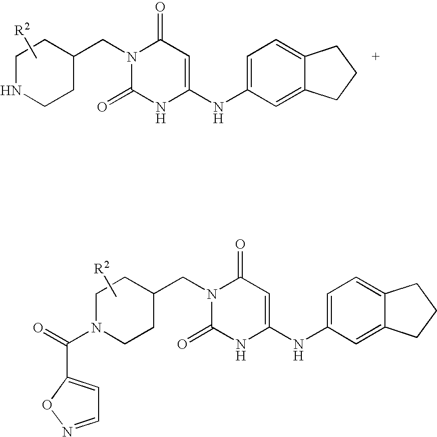

The compounds of the invention show a valuable range of pharmaceutical and pharmacokinetic effects which could not have been predicted. They are therefore suitable for use as medicaments for the treatment and/or prophylaxis of diseases in humans and animals.

The compounds of the invention are particularly effective against bacteria and bacteroid microorganisms, especially Gram-positive bacteria. They are therefore particularly well-suited for the prophylaxis and chemotherapy of local and, where appropriate, systemic infections caused by these pathogens in human and veterinary medicine.

It is possible for example to treat and/or prevent local and/or systemic diseases caused by the following pathogens or by mixtures of the following pathogens:

Gram-positive cocci, e.g. staphylococci (Staph. aureus, Staph. epidermidis) and streptococci (Strept. agalactiae, Strept. faecalis, Strept. pneumoniae, Strept. pyogenes), and strictly anaerobic bacteria such as, for example, clostridium, also mycoplasmas (M. pneumoniae, M. hominis, M. urealyticum).

The above list of pathogens is merely by way of example and by no means to be regarded as restrictive. Examples of diseases which are caused by the mentioned pathogens or mixed infections, and can be prevented, improved or cured by the compounds of the invention, and which may be mentioned are:

Infectious diseases in humans such as, for example, septic infections, bone and joint infections, skin infections, postoperative wound infections, abscesses, phlegmon, wound infections, infected burns, burn wounds, infections in the oral region, infections after dental operations, septic arthritis, mastitis, tonsillitis, genital infections and eye infections.

Apart from humans, it is also possible to treat bacterial infections in other species. Examples which may be mentioned are:

Pig: sepsis, metritis-mastitis-agalactiae syndrome, mastitis;

Ruminants (cattle, sheep, goats): sepsis, bronchopneumonia, mycoplasmosis, genital infections;

Horse: bronchopneumonias, puerperal and postpuerperal infections;

Dogs and cats: bronchopneumonia, dermatitis, otitis, urinary tract infections, prostatitis;

Poultry (chickens, turkeys, quail, pigeons, ornamental birds and others): mycoplasmosis, chronic airway diseases, psittacosis.

It is likewise possible to treat bacterial diseases in the rearing and management of productive and ornamental fish, in which case the antibacterial spectrum is extended beyond the pathogens mentioned above to further pathogens such as, for example, brucella, campylobacter, listeria, erysipelothris, nocardia.

The active ingredient may have systemic and/or local effects. For this purpose, it can be administered in a suitable manner such as, for example, by the oral, parenteral, pulmonary, nasal, sublingual, lingual, buccal, rectal, transdermal, conjunctival or otic route, or as implant. Parenteral administration is preferred.

The active ingredient can be administered in suitable administration forms for these administration routes.

Administration forms suitable for oral administration are known ones which deliver the active ingredient in a rapid and/or modified way, such as, for example, tablets (uncoated and coated tablets, e.g. tablets provided with coatings resistant to gastric juice), capsules, sugar-coated tablets, granules, pellets, powders, emulsions, suspensions and solutions.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous or intraperitoneal). Administration forms suitable for parenteral administration include preparations for injection and infusion in the form of solutions, suspensions, emulsions, lyophilisates and sterile powders. Intravenous administration is preferred.

Examples suitable for the other administration routes are medicinal forms for inhalation (inter alia powder inhalers, nebulizers), nasal drops/solutions, sprays; tablets for lingual, sublingual or buccal administration, or capsules, suppositories, preparations for the eyes and ears, vaginal capsules, aqueous suspensions (lotions, shaking mixtures), lipophilic suspensions, ointments, creams, milk, pastes, dusting powders or implants.

The active ingredients can be converted in a manner known per se into the administration forms listed. This takes place with use of inert nontoxic, pharmaceutically suitable excipients. These include inter alia carriers (e.g. microcrystalline cellulose), solvents (e.g. liquid polyethylene glycols), emulsifiers (e.g. sodium dodecyl sulfate), dispersants (e.g. polyvinylpyrrolidone), synthetic and natural biopolymers (e.g. albumin), stabilizers (e.g. antioxidants such as ascorbic acid), colorants (e.g. inorganic pigments such as iron oxides) or masking tastes and/or odors.

It has generally proved advantageous for parenteral administration to administer amounts of about 0.001 to 10 mg/kg, preferably about 0.01 to 1 mg/kg, of body weight to achieve effective results. On oral administration the amount is about 0.01 to 500 mg/kg, preferably about 1 to 10 mg/kg, of body weight.

It may nevertheless be necessary where appropriate to deviate from the amounts mentioned, in particular as a function of the body weight, administration route, individual response to the active ingredient, type of preparation and time or interval over which administration takes place.

Particular preference is given to parenteral, especially intravenous, administration, e.g. as iv bolus injection (i.e. as single dose, e.g. by syringe), short infusion (i.e. infusion over a period of up to one hour) or long infusion (i.e. infusion over a period of more than one hour). The administered volume may in these cases be, depending on the specific conditions, between 0.5 to 30, in particular 1 to 20 ml on iv bolus injection, between 25 to 500, in particular 50 to 250 ml on short infusion and between 50 to 1000, in particular 100 to 500 ml on long infusion. It may for this purpose be advantageous for the active ingredient to be provided in solid form (e.g. as lyophilisate) to be dissolved in the dissolving medium only directly before administration.

(It is necessary in these cases for the administration forms to be sterile and pyrogen-free. They may be based on aqueous or mixtures of aqueous and organic solvents.

These include, for example, aqueous solutions, mixtures of aqueous and organic solvents (especially ethanol, polyethylene glycol (PEG) 300 or 400), aqueous solutions containing cyclodextrins or aqueous solutions containing emulsifiers (surface-active solubilizers, e.g. lecithin or Pluronic F 68, Solutol HS15, Cremophor). Aqueous solutions are preferred in this connection.

Formulations suitable for parenteral administration are those which are substantially isotonic and euhydric, e.g. those with a pH between 3 and 11, in particular 6 and 8, especially around 7.4.

Solutions for injection are packaged in suitable containers made of glass or plastic, e.g. in vials. These may have a volume of from 1 to 1000, in particular 5 to 50 ml. The solution can be removed directly therefrom and administered. In the case of a lyophilisate, it is dissolved in the vial by injecting a suitable solvent and is then removed.

Solutions for infusion are packaged in suitable containers made of glass or plastic, e.g. in bottles or collapsible plastic bags. These may have a volume of from 1 to 1000, in particular 50 to 500, ml.

The percentage data in the following tests and examples are, unless indicated otherwise, percentages by weight; parts are parts by weight. Solvent ratios, dilution ratios and concentration data for liquid/liquid solutions are in each case based on volume.

### A. EXAMPLES

Abbreviations:

aq. aqueous
Bn benzyl
BOP benzotriazol-1-yloxytris(dimethylamino)phosphonium hexafluorophosphate
DMSO dimethyl sulfoxide
DMF dimethylformamide
eq. equivalent
ESI electrospray ionization (in MS)
h hour
HPLC high pressure, high performance liquid chromatography
LC-MS coupled liquid chromatography-mass spectroscopy
MS mass spectroscopy
NMR nuclear magnetic resonance spectroscopy
Pd/C palladium/carbon
RP-HPLC reverse phase HPLC
RT room temperature
Rₜ retention time (in HPLC)
   HPLC and LC-MS Methods:
   Method 1: Instrument Micromass Quattro LCZ

Column: Symmetry C18, 50 mm×2.1 mm, 3.5 μm; temperature: 40° C.; flow rate=0.5 mlmin⁻¹; eluent-A=CH₃CN+0.1% formic acid, eluent B=water+0.1% formic acid; gradient 0.0 min 10% A→4 min 90% A→6 min 90% A

Method 2: Instrument Micromass Platform LCZ

Column: Symmetry C18, 50 mm×2.1 mm, 3.5 μm; temperature: 40° C.; flow rate=0.5 mlmin⁻¹; eluent A=CH₃CN+0.1% formic acid, eluent B=water+0.1% formic acid; gradient 0.0 min 10% A→4 min 90% A→6 min 90% A

Method 3: Instrument: Finnigan MAT 900S, TSP: P4000, AS3000, UV3000HR

Column: symmetry C 18, 150 mm×2.1 mm, 5.0 μm; eluent C: water, eluent B: water+0.3 g of 35% strength HCl, eluent A: acetonitrile; gradient: 0.0 min 2% A→2.5 min 95% A→5 min 95% A; oven: 70° C.; flow rate: 1.2 mlmin; UV detection: 210 nm

Starting Compounds:

### Example I

### 1-[(Benzyloxy)methyl]-6-chloro-2,4(1H,3H)pyrimidinedione

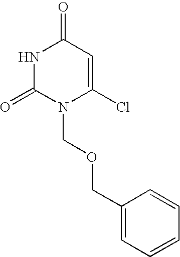

45 g (307.09 mmol) of chlorouracil are mixed with 600. ml of dimethylformamide and cooled to 0° C. 3.22 g (405.46 mmol) of lithium hydride are cautiously added (highly exothermic), followed by stirring for 10 minutes. Then, 62.25 g (317.17 mmol) of benzyl chloromethyl ether are added dropwise over the course of 10 minutes. This is followed by stirring at 0° C. for 90 minutes. The reaction solution is mixed with 1000 ml of 2% strength sodium hydroxide solution and extracted with 1000 ml of toluene. The toluene phase is washed once with 100 ml of 2% strength sodium hydroxide solution. The combined aqueous phases are adjusted to pH 3 with IN sulfuric acid. The precipitated solid is filtered off and dried under high vacuum. 57.1 g (70% of theory) of product are obtained.

¹H-NMR (200 MHz, CDCl3): δ=4.69 (s, 2H), 5.54 (s, 2H), 5.87 (s, 1H), 7.35 (m, 5H), 8.45 (s, 1H).

### Example II

### tert-Butyl 4-(hydroxymethyl)-1-piperidinecarboxylate

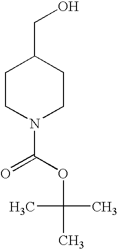

12.1 g (105.06 mmol) of 4-(hydroxymethyl)piperidine are dissolved together with 0.31 g (2.5 mmol) of 4-dimethylaminopyridine and 32.22 ml (231.13 mmol) of triethylamine in 305 ml of dichloromethane. The mixture is cooled to 0° C., and 24.07 g (110.31 mmol) of di-tert-butyl pyrocarbonate are added in portions. This is followed by stirring at room temperature for 50 hours. The reaction solution is mixed with 25 ml of water. The phases are separated, and the organic phase is washed once with 100 ml of 1M hydrochloric acid and once with 50 ml of sodium chloride solution. It is dried over sodium sulfate and concentrated in vacuo. 20 g (92% of theory) of product are obtained.

LC-MS (method 3): Rₜ=2.13 min

MS (ESIpos): m/z=216 (M+H)⁺

### Example III

### tert-Butyl 4-{[3-[(benzyloxy)methyl]-4-chloro-2,6-dioxo-3,6-dihydro-1(2H)-pyrimidinyl]methyl}-1-piperidinecarboxylate

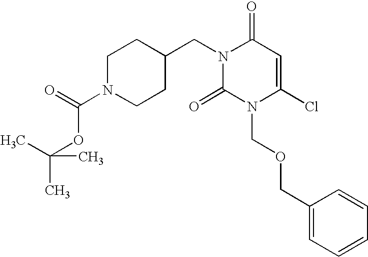

5 g (23.22 mmol) of the compound from example I, 6.19 g (23.22 mmol) of the compound from example II and 6.7 g (25.55 mmol) of triphenylphosphine are mixed in 153 ml of tetrahydrofuran. The mixture is cooled to 0° C., and 4.02 ml (25.55 mmol) of diethyl azodicarboxylate are added dropwise over the course of 10 minutes. This is followed by stirring at room temperature for 72 hours. The reaction solution is concentrated in vacuo and purified on silica gel 60.

7.43 g (69% of theory) of product are obtained.

LC-MS (method 2): Rₜ=4.7 min

MS (ESIpos): m/z=464 (M+H)⁺

### Example IV

### 6-Chloro-3-(4-piperidinylmethyl)-2,4(1H,3H)pyrimidinedione

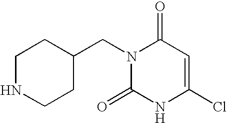

9.85 g (21.23 mmol) of the compound from-example III are mixed with 161 ml of trifluoroacetic acid and heated to reflux. After 2 hours, the reaction solution is cooled and concentrated in vacuo. It is taken up once more in methanol and again concentrated in vacuo. The residue is recrystallized from 30 ml of ethyl acetate. 5.43 g (99% of theory) of product are obtained.

LC-MS (method 1): Rₜ=0.32 min

MS (ESIpos): m/z=244 (M+H)⁺

### Example V

### 6-(2,3-Dihydro-1H-inden-5-ylamino)-3-(4-piperidinylmethyl)-2,4(1H,3H)pyrimidinedione

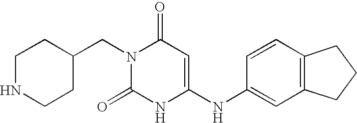

0.77 g (3.18 mmol) of the compound from example IV are mixed with 0.93 g (7 mmol) of 5-aminoindane and heated to 150° C. After 2 hours, the reaction solution is cooled. 10 ml of dichloromethane and 5 ml of methanol are added, and this mixture is added with stirring to 50 ml of diethyl ether. The precipitate is filtered off and dried under high vacuum.

0.667 g (61% of theory) of product is obtained.

LC-MS (method 1): Rₜ=2.37 min

MS (ESIpos): m/z=341 (M+H)⁺

### Example VI

### 6-[(3-Ethyl-4-methylphenyl)amino]-3-(4-piperidinylmethyl)-2,4(1H,3H)pyrimidinedione

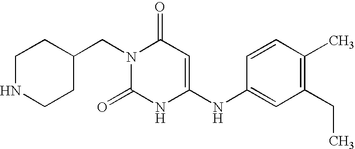

Preparation takes place as for example V from 1 g (4.104 mmol) of the compound from example IV, 0.845 g (4.924 mmol) of 3-ethyl-4-methylaniline hydrochloride and 1.57 ml (9.02 mmol) of N,N-diisopropylethylamine.

0.498 g (35% of theory) of product is obtained.

LC-MS (method 1): Rₜ=2.47 min

MS (ESIpos): m/z=343 (M+1H)⁺

### PREPARATION EXAMPLES

### Example 1

### 6-(2,3-Dihydro-1H-inden-5-ylamino)-3-({1-[(5-methyl-4-isoxazolyl)carbonyl]-4-piperidinyl}methyl)-2,4(1H,3H)pyrimidinedione

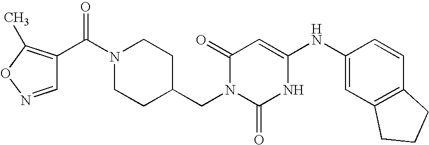

133.9 mg (0.39 mmol) of the compound from example V are suspended together with 50 mg (0.39 mmol) of 5-methylisoxazole-4-carboxylic acid and 225.19 mg (0.43 mmol) of 1-benzotriazolyloxytripyrrolidinephosphonium hexafluorophosphate in 1.5 ml of tetrahydrofuran. 55.93 mg (0.43 mmol) of N,N-diisopropylethylamine are added, followed by stirring at room temperature for 24 hours. 0.5 ml of water is added to the reaction solution. After 15 minutes, it is filtered through an Extrelut/silica gel cartridge. 3 ml of methanol/dichloromethane in the ratio 2:1 are used for washing. The filtrate is concentrated in vacuo. The residue is taken up in ethyl acetate and washed twice with sodium bicarbonate solution. Sodium sulfate is used for drying, and the solution is concentrated in vacuo. Preparative HPLC is used for purification.

65 mg (37% of theory) of product are obtained.

LC-MS (method 2): Rₜ=3.57 min

MS (ESIpos): m/z=450 (M+H)⁺

The examples listed in the table below can be prepared from the appropriate starting compounds in analogy to the methods described above.

| Example | Structure | Analytical data |
|---|---|---|
| 2 | 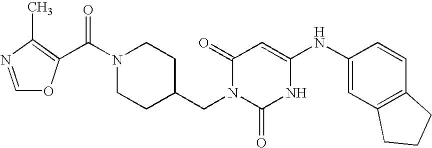 | LC-MS (method 2): Rt = 3.43 min MS (ESIpos): m/z = 450 (M + H) |
| 3 | 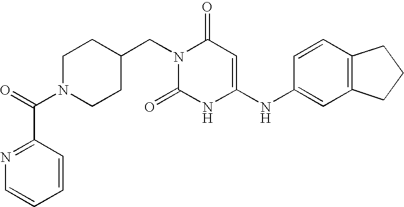 | LC-MS (method 2): Rt = 3.39 min MS (ESIpos): m/z = 446 (M + H) |
| 4 | 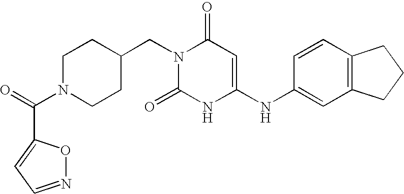 | LC-MS (method 1): Rt = 3.41 min MS (ESIpos): m/z = 436 (M + H) |
| 5 | 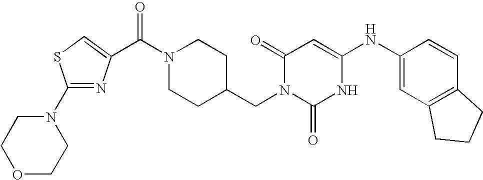 | LC-MS (method 1): Rt = 3.48 min MS (ESIpos): m/z = 537 (M + H) |
| 6 | 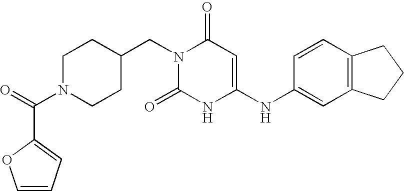 | LC-MS (method 1): Rt = 3.5 min MS (ESIpos): m/z = 435 (M + H) |
| 7 | 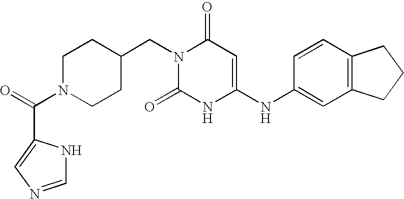 | LC-MS (method 1): Rt = 2.61 min MS (ESIpos): m/z = 435 (M + H) |
| 8 | 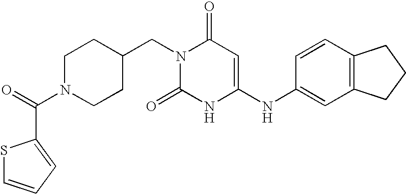 | LC-MS (method 1): Rt = 3.63 min MS (ESIpos): m/z = 451 (M + H) |
| 9 | 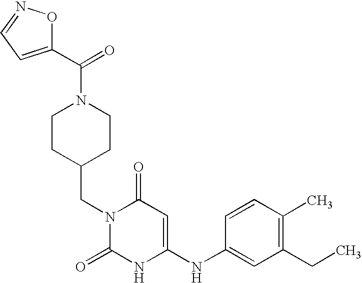 | LC-MS (method 1): Rt = 3.51 min MS (ESIpos): m/z = 438 (M + H) |
| 10 | 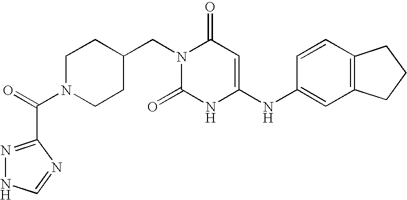 | LC-MS (method 2): Rt = 3.13 min MS (ESIpos): m/z 436 (M + H) |
| 11 | 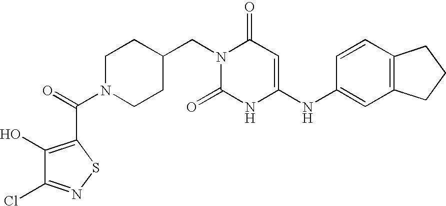 | LC-MS (method 2): Rt = 3.5 min MS (ESIpos): m/z = 502 (M + H) |
| 12 | 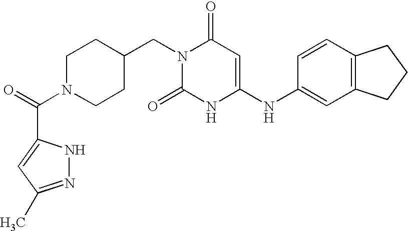 | LC-MS (method 2): Rt = 3.33 min MS (ESIpos): m/z = 449 (M + H) |
| 13 | 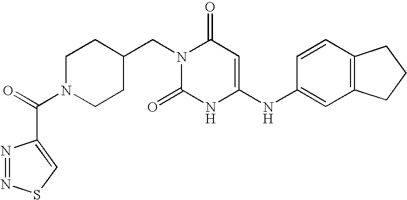 | LC-MS (method 2): Rt = 3.45 min MS (ESIpos): m/z = 453 (M + H) |
| 14 | 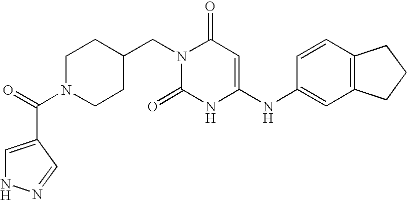 | LC-MS (method 2): Rt = 3.21 min MS (ESIpos): m/z = 435 (M + H) |
| 15 | 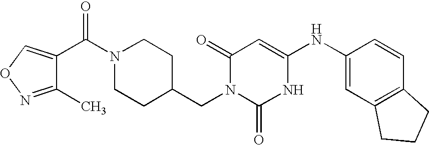 | LC-MS (method 2): Rt = 3.49 min MS (ESIpos): m/z = 450 (M + H) |
| 16 | 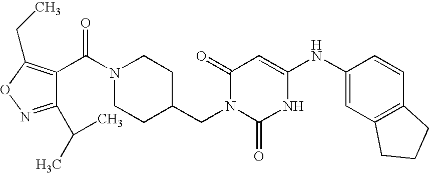 | LC-MS (method 2): Rt = 3.97 min MS (ESIpos): m/z = 506 (M + H) |
| 17 | 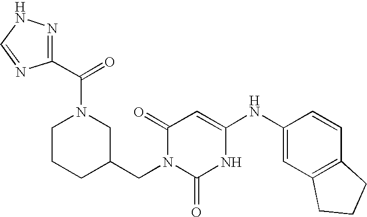 | LC-MS (method 2): Rt = 3.13 min MS (ESIpos): m/z = 436 (M + H) |
| 18 | 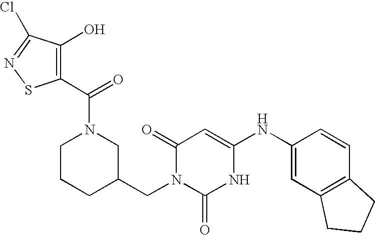 | LC-MS (method 2): Rt = 3.49 min MS (ESIpos): m/z = 502 (M + H) |
| 19 | 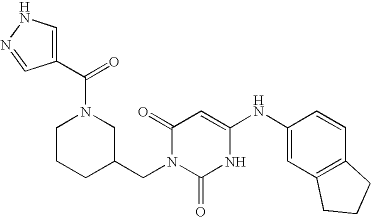 | LC-MS (method 2): Rt = 3.21 min MS (ESIpos): m/z = 435 (M + H) |
| 20 | 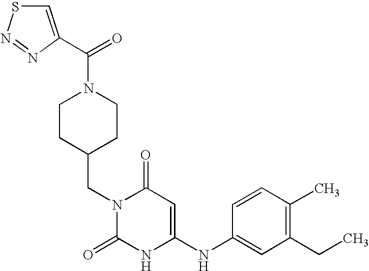 | LC-MS (method 1): Rt = 3.48 min MS (ESIpos): m/z = 455 (M + H) |
| 21 | 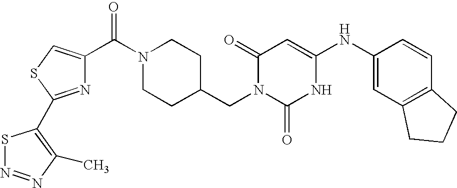 | LC-MS (method 2): Rt = 3.79 min MS (ESIpos): m/z = 550 (M + H) |
| 22 | 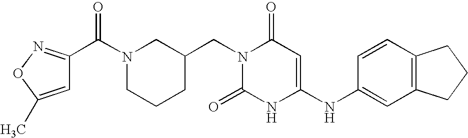 | |
| 23 | 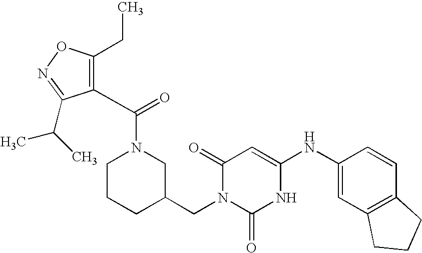 | LC-MS (method 1): Rt = 3.85 min MS (ESIpos): m/z = 506 (M + H) |
| 24 | 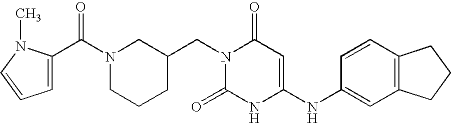 | LC-MS (method 2): Rt = 3.69 min MS (ESIpos): m/z = 448 (M + H) |
| 25 | 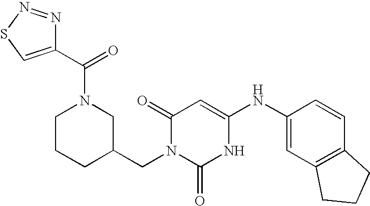 | LC-MS (method 1): Rt = 3.41 min MS (ESIpos): m/z = 453 (M + H) |
| 26 | 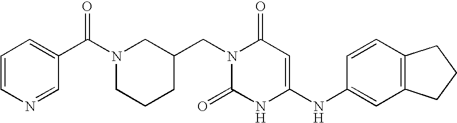 | LC-MS (method 1): Rt = 3.16 min MS (ESIpos): m/z = 446 (M + H) |
| 27 | 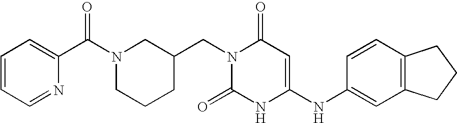 | LC-MS (method 1): Rt = 3.29 min MS (ESIpos): m/z = 446 (M + H) |
| 28 | 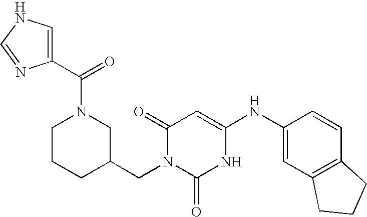 | LC-MS (method 2): Rt = 2.71 min MS (ESipos): m/z = 435 (M + H) |
| 29 | 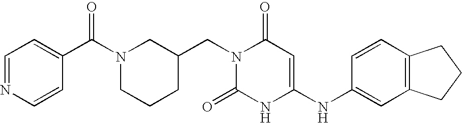 | LC-MS (method 1): Rt = 3.05 min MS (ESIpos): m/z = 446 (M + H) |
| 30 | 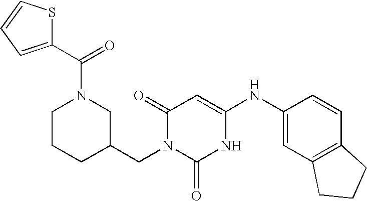 | LC-MS (method 1): Rt = 3.65 min MS (ESIpos): m/z = 451 (M + H) |
| 31 | 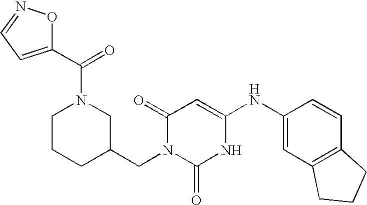 | LC-MS (method 1): Rt = 3.45 min MS (ESIpos): m/z = 436 (M + H) |
| 32 | 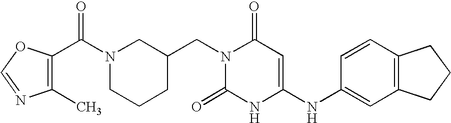 | LC-MS (method 1): Rt = 3.37 min MS (ESIpos): m/z = 450 (M + H) |
| 33 | 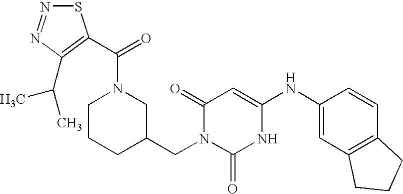 | LC-MS (method 1): Rt = 3.85 min MS (ESIpos): m/z = 495 (M + H) |
| 34 | 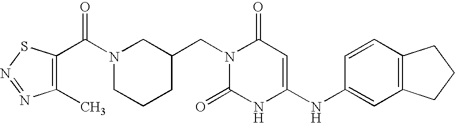 | LC-MS (method 1): Rt = 3.57 min MS (ESIpos): m/z = 467 (M + H) |
| 35 | 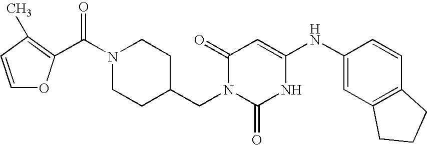 | LC-MS (method 1): Rt = 3.72 min MS (ESIpos): m/z = 449 (M + H) |
| 36 | 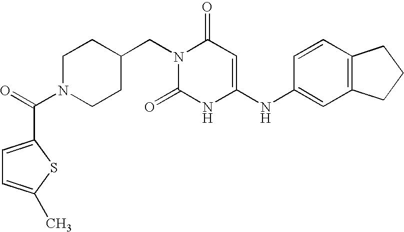 | LC-MS (method 1): Rt = 3.84 min MS (ESIpos): m/z = 465 (M + H) |
| 37 | 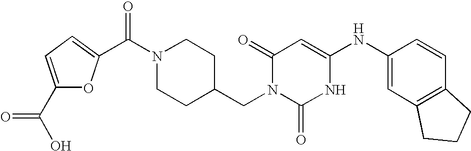 | LC-MS (method 1): Rt = 3.51 min MS (ESIpos): m/z = 479 (M + H) |
| 38 | 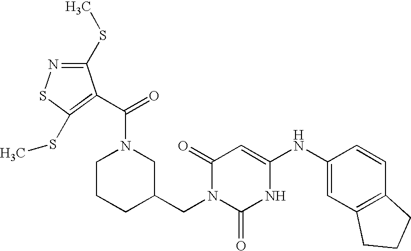 | LC-MS (method 2): Rt = 3.95 min MS (ESIpos): m/z = 544 (M + H) |
| 39 | 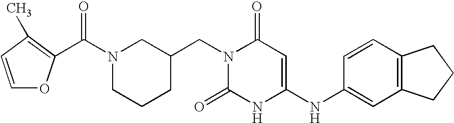 | LC-MS (method 2): Rt = 3.74 min MS (ESIpos): m/z = 449 (M + H) |
| 40 | 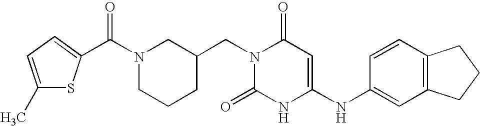 | LC-MS (method 2): Rt = 3.86 min MS (ESIpos): m/z = 465 (M + H) |
| 41 | 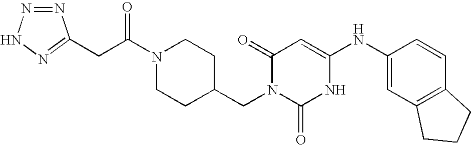 | LC-MS (method 1): Rt = 3.26 min MS (ESIpos): m/z = 451 (M + H) |
| 42 | 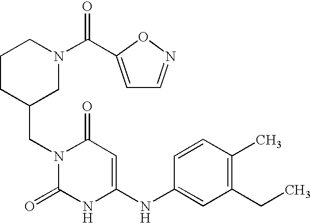 | LC-MS (method 1): Rt = 3.59 min MS (ESIpos): m/z = 438 (M + H) |
| 43 | 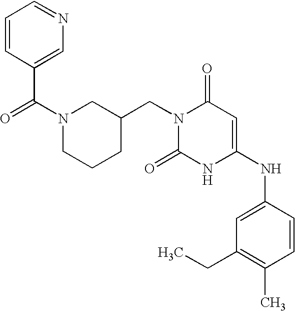 | LC-MS (method 1): Rt = 3.29 min MS (ESIpos): m/z = 448 (M + H) |
| 44 | 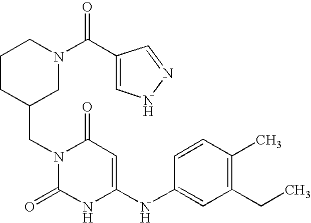 | LC-MS (method 1): Rt = 3.27 min MS (ESIpos): m/z = 437 (M + H) |
| 45 | 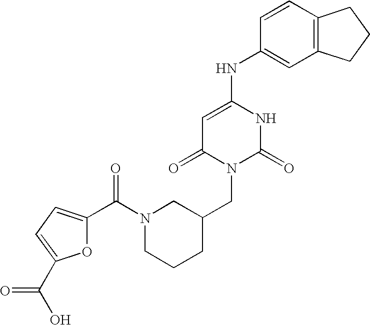 | LC-MS (method 1): Rt = 3.52 min MS (ESIpos): m/z = 479 (M + H) |
| 46 | 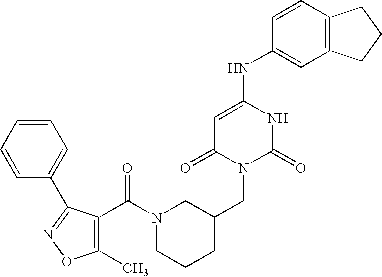 | LC-MS (method 2): Rt = 4.11 min MS (ESIpos): m/z = 526 (M + H) |
| 47 | 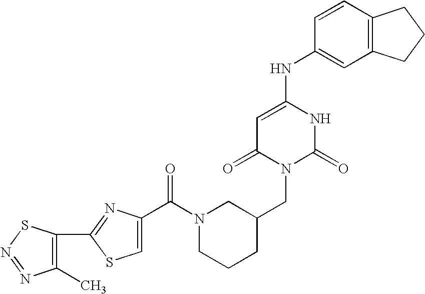 | LC-MS (method 1): Rt = 3.68 min MS (ESIpos): m/z = 550 (M + H) |
| 48 | 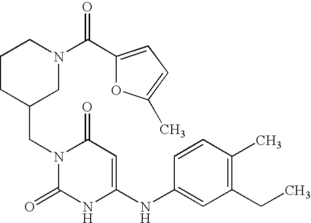 | LC-MS (method 1): Rt = 3.78 min MS (ESIpos): m/z = 451 (M + H) |

### B. ASSESSMENT OF THE PHARMACOLOGICAL ACTIVITY

In Vitro Effect

The in vitro effect of the compounds of the invention can be shown in the following assays:

Cloning, Expression and Purification of Pol III from S. aureus

To clone polC with an N-terminal His tag, the structural gene polC is amplified from S. aureus genomic DNA with the aid of PCR. The primers SAPol 31 5′-GCGCCATATGGACAGAGCAACAAAAATTTAA-3′ and SAPolrev 5′-GCGCGGATCCTTACATATCAAATATCGAAA-3′ are used to introduce the NdeI and BamHI restriction cleavage sites respectively in front of and behind the amplified gene. After the PCR product which is 4300 bp in size has been digested with NdeI and BamHI, it is ligated into the vector pET15b (Novagen, USA), which has likewise been digested with NdeI and BamHI, and transformed into E. coli XL-1 Blue.

After transformation into E. coli BL21(DE3), the cells are cultivated for expression of PolC at 30° C. in LB medium with 100 μg/ml ampicillin until the OD_{595 nm} is 0.5, cooled to 18° C. and, after addition of 1 mM IPTG, incubated for a further 20 hours. The cells are harvested by centrifugation, washed once in PBS with 1 mM PMSF and taken up in 50 mM NaH₂PO₄ pH 8.0, 10 mM imidazole, 2 mM β-mercaptoethanol, 1 mM PMSF, 20% glycerol. The cells are disrupted using a French press at 12,000 psi, the cell detritus is removed by centrifugation (27,000 TNR×g, 120 min, 4° C.) and the supernatant is stirred with an appropriate amount of Ni-NTA-agarose (from Quiagen, Germany) at 4° C. for 1 hour. After the gel matrix has been packed into a column it is washed with 50 mM NaH₂PO₄ pH 8.0, 2 mM β-mercaptoethanol, 20 mM imidazole, 10% glycerol, and the purified protein is then eluted with the same buffer containing 100 mM imidazole. The purified protein is mixed with 50% glycerol and stored at −20° C.

Determination of the IC₅₀ for Polymerase III

The activity of PolC is measured in an enzymatically coupled reaction, with the pyrophosphate formed during the polymerization being converted with the aid of ATP sulfurylase into ATP, which is detected using firefly luciferase. The reaction mixture contains, in a final volume of 50 μl, 50 mM Tris/Cl pH 7.5; 5 mM DTT, 10 mM MgCl₂, 30 mM NaCl, 0.1 mg/ml BSA, 10% glycerol, 20 μM each dATP, dTTP, dCTP, 2U/ml activated calf thymus DNA (from Worthington, USA), 20 μM APS and 0.06 mM luciferin. The reaction is started by adding purified PolC in a final concentration of ˜2 nM and is incubated at 30° C. for 30 min. The amount of pyrophosphate formed is then converted into ATP by adding ATP sulfurylase (Sigma, USA) in a final concentration of 5 nM and incubating at 30° C. for 15 min. After addition of 0.2 nM firefly luciferase, the luminescence is measured in a luminometer for 60 s. The IC₅₀ is reported as the concentration of an inhibitor which leads to 50% inhibition of the enzymic activity of PolC.

TABLE A

| Example No. | IC₅₀ (μM) |
|---|---|
| 9 | 0.14 |
| 13 | 0.09 |
| 17 | 0.34 |
| 21 | 0.11 |
| 26 | 0.22 |
| 39 | 0.33 |

Determination of the Minimum Inhibitory Concentrations (MIC) for a Number of Microbes

The MIC values for various bacterial strains (S. aureus, S. pneumoniae, E. faecalis) are carried out using the microdilution method in BHI broth. The bacterial strains are cultured overnight in BHI broth (staphylococci) or BHI broth+10% bovine serum (streptococci, enterococci). The test substances are tested in a concentration range from 0.5 to 256 μg/ml. After serial dilution of the test substances, the microtiter plates are inoculated with the test microbes. The microbe concentration is about 1×10⁶ microbes/ml of suspension. The plates are incubated at 37° C. under 8% CO₂ (for streptococci, enterococci) for 20 h. The MIC is recorded as the lowest concentration at which visible growth of the bacteria is completely inhibited.

In Vivo Effect

The suitability of the compounds of the invention for treating bacterial infections can be shown in the following animal models:

Systemic Infection with S. aureus 133

S. aureus 133 cells are cultured overnight in BH broth. The overnight culture is diluted 1:100 in fresh BH broth and amplified for 3 hours. The bacteria, which are in the logarithmic phase of growth, are spun down and washed 2× with buffered physiological saline (303). Then a cell suspension is adjusted in a photometer (model LP 2W from Dr. Lange, Germany) to an extinction of 50 units in 303. After a dilution step (1:15), this suspension is mixed 1:1 with a 10% strength mucin suspension. 0.25 ml of this infection solution is administered ip per 20 g mouse. This corresponds to a cell count of approximately 1×10E⁶ microbes/mouse. The ip therapy takes place 30 minutes after the infection. Female CFW1 mice are used for the infection experiment. The survival of the animals is recorded for 6 days.

### C. EXEMPLARY EMBODIMENTS OF PHARMACEUTICAL COMPOSITIONS

The substances of the invention can be converted into pharmaceutical preparations in the following ways:

Tablet:

Composition:

100 mg of the compound of example 1, 50 mg of lactose (monohydrate), 50 mg of corn starch (native), 10 mg of polyvinylpyrolidone (PVP 25) (from BASF, Ludwigshafen, Germany) and 2 mg of magnesium stearate.

Tablet weight 212 mg. Diameter 8 mm, radius of curvature 12 mm.

Production:

A mixture of active ingredient, lactose and starch is granulated with a 5% strength solution (m/m) of the PVP in water. The granules are dried and then mixed with the magnesium stearate for 5 min. This mixture is compressed using a conventional tablet press (see above for format of the tablet). A compressive force of 15 kN is used as guideline for the compression.

Suspension Which Can Be Administered Orally:

Composition:

1000 mg of the compound of example 1, 1000 mg of ethanol (96%), 400 mg of Rhodigel (xanthan gum from FMC, Pennsylvania, USA) and 99 g of water. 10 ml of oral suspension correspond to a single dose of 100 mg of the compound of the invention.

Production:

The Rhodigel is suspended in ethanol, and the active ingredient is added to the suspension. The water is added while stirring. The mixture is stirred for about 6 h until the swelling of the Rhodigel is complete.

Solution Which Can Be Administered Intravenously:

Composition:

1 mg of the compound of example 1, 15 g of polyethylene glycol 400 and 250 g of water for injections.

Production:

The compound of example 1 is dissolved together with polyethylene glycol 400 in the water with stirring. The solution is sterilized by filtration (pore diameter 0.22 μm) and used to fill heat-sterilized infusion bottles under aseptic conditions. These are closed with infusion stoppers and crimped caps.

1. A compound of the formula 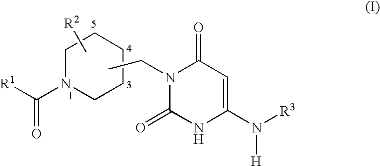
in which
R¹ is heteroaryl,
where heteroaryl may be substituted by 0, 1, 2 or 3 substituents selected independently of one another from the group consisting of alkyl, alkoxy, alkylthio, halogen, alkanoyl, hydroxy, trifluoromethyl, trifluoromethoxy, nitro, amino, alkylamino, alkanoylamino, cyano, carboxy, cycloalkyl, heterocyclyl, aryl and optionally methyl-substituted heteroaryl,
R² is hydrogen or alkyl,
R³ is a substituent of the following formula 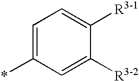
in which
R³⁻¹ and R³⁻² are selected independently of one another from the group consisting of alkyl, alkenyl, alkynyl, alkylthio, cycloalkyl and halogen, or
R³⁻¹ and R³⁻² form together with the carbon atoms to which they are bonded a cycloalkyl or heterocyclyl ring which is optionally substituted by up to 3 halogen.

2. A compound of the formula (I) as claimed in claim 1, in which
the uracil ring is linked via positions 3, 4 or 5 to the piperidine ring,
R¹ is heteroaryl,
where heteroaryl may be substituted by 0, 1, 2 or 3 substituents selected independently of one another from the group consisting of alkyl, alkoxy, alkylthio, halogen, hydroxy, carboxy, heterocyclyl and aryl,
R² is hydrogen or alkyl,
R³ is a substituent of the following formula 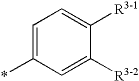
in which
R³⁻¹ and R³⁻² are selected independently of one another from the group consisting of C₁-C₆-alkyl, C₂-C₆-alkenyl, C₂-C₆-alkynyl, C₁-C₆-alkylthio, C₃-C₆-cycloalkyl and halogen, or
R³⁻¹ and R³⁻² form together with the carbon atoms to which they are bonded a C₃-C₆-Cycloalkyl or heterocyclyl ring which is optionally substituted by up to 3 halogen.

3. A compound of the formula (I) as claimed in claim 1 or 2, in which
the uracil ring is linked via positions 3, 4 or 5 to the piperidine ring,
R¹ is heteroaryl,
where heteroaryl may be substituted by 0, 1 or 2 substituents selected independently of one another from the group consisting of alkyl, halogen and carboxy,
R² is hydrogen, and
R³ is a substituent of the following formula 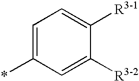
in which
R³⁻¹ and R³⁻² are selected independently of one another from the group consisting of methyl, ethyl, fluorine and chlorine, or
R³⁻¹ and R³⁻² form together with the carbon atoms to which they are bonded a C₅-cycloalkyl ring which is optionally substituted by up to 2 substituents selected independently of one another from the group consisting of chlorine or fluorine.

4. A compound of the formula (I) as claimed in claim 1 or 2, in which the uracil ring is linked via positions 3, 4 or 5 to the piperidine ring.

5. A compound of the formula (I) as claimed in any of claims 1 to 4, in which R¹ is a 5- to 6-membered heteroaryl radical.

6. A compound of the formula (I) as claimed in claim 1, 2, 4 or 5, in which R² is hydrogen.

7. A compound of the formula (I) as claimed in any of claims 1 to 6, in which R³ is selected from the group of

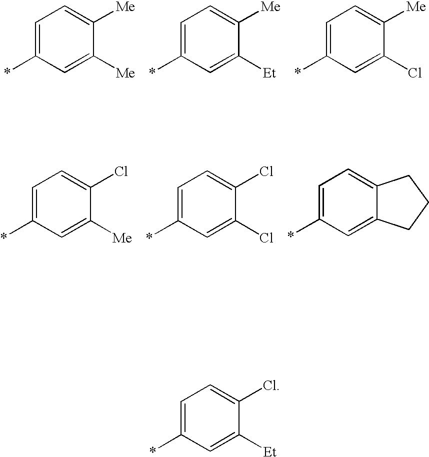

8. A process for preparing compounds of the formula (I) by reacting compounds of the formula 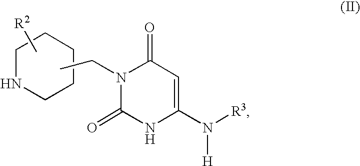
in which
R² and R³ have the meaning indicated in claim 1,
with compounds of the general formula 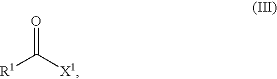
in which
R¹ has the meaning indicated in claim 1, and
X¹ is halogen, preferably chlorine or bromine, or hydroxy.

9. A compound as claimed in any of claims 1 to 7 for the treatment and/or prophylaxis of diseases.

10. A medicament comprising at least one compound as claimed in any of claims 1 to 7 in combination with at least one pharmaceutically suitable, pharmaceutically acceptable carrier or excipient.

11. The use of compounds as claimed in any of claims 1 to 7 for producing a medicament for the treatment and/or prophylaxis of bacterial infections.

12. A medicament as claimed in claim 10 for the treatment and/or prophylaxis of bacterial infections.

13. A method for controlling bacterial infections in humans and animals by administering an antibacterially effective amount of at least one compound as claimed in any of claims 1 to 7.
